(19) United States
(12) Patent Application Publication
(10) Pub. No.: US 2025/0352515 A1
(43) Pub. Date: Nov. 20, 2025

(54) TOPICAL FORMULATIONS

(71) Applicant: OCuSOFT, Inc., Rosenberg, TX (US)

(72) Inventors: Paula Hooper, Rosenberg, TX (US); Troy Smith, Rosenberg, TX (US); Nat Adkins, Rosenberg, TX (US)

(21) Appl. No.: 19/285,203

(22) Filed: Jul. 30, 2025

Related U.S. Application Data
(63) Continuation-in-part of application No. 19/069,051, filed on Mar. 3, 2025.
(60) Provisional application No. 63/561,066, filed on Mar. 4, 2024.

(51) Int. Cl. A61K 31/4164; A61K 9/06; A61K 31/7048; A61K 36/61; A61P 29/00; A61P 33/14

(52) U.S. Cl. CPC A61K 31/4164; A61K 9/06; A61K 31/7048; A61K 36/61; A61P 29/00; A61P 33/14

(57) ABSTRACT

A topical formulation, comprising: an ointment base; and a mixture of active pharmaceutical ingredients (APIs) contained in the ointment base, the mixture of APIs consisting of: Metronidazole; Ivermectin; and Melaleuca alternifolia (tea tree) oil. The APIs are about 0.12%-6% w/w and the ointment base is about 94% to 99% w/w of the topical formulation. The formulation can further include enhancers for the APIs, such as, moisturizers and anti-oxidants. The formulation is configured as an anti-parasitic, anti-inflammatory and anti-microbial formulation.

### CROSS-REFERENCE TO RELATED APPLICATION

This application is a continuation in part of U.S. patent application Ser. No. 19/069,051, filed on Mar. 3, 2025, which claims the benefit of Provisional U.S. Patent Application No. 63/561,066, filed Mar. 4, 2024, entitled “Medicated Eyelid Scrub Composition”, the entire content and disclosure of which, both express and implied, is incorporated herein by reference.

### TECHNICAL FIELD

The present invention is a topical formulation for ophthalmological and dermatological use. In particular, the formulation includes a mixture of three active pharmaceutical ingredients contained in an ointment base.

### BACKGROUND

The eyelids play a crucial role in maintaining overall ocular health by shielding the eyes from external threats such as airborne contaminants or approaching objects. They contain several glands, including the lacrimal and meibomian glands, which produce a vital layer of tear film that keeps the eyes moist and healthy. Every time an individual blinks, the eyelids create a fresh tear film that lubricates the eye's surface and clears out foreign bodies.

However, eyelids are susceptible to various issues that can cause discomfort, particularly for contact lens wearers, and can lead to severe complications. One such issue is blepharitis, a common chronic inflammation characterized by scaly crust on the lid margins. Blepharitis can be caused by a bacterial infection, allergies, or seborrhea of the face and scalp. Regular cleaning of the eyelids is essential to remove excess oil, debris, and dead skin that can exacerbate the problem.

Blepharitis can also lead to other complications such as an internal hordeolum, a bacterial infection of the skin at the edge of the lid, or an external hordeolum, commonly known as a stye, which is an infection of the meibomian glands. Styes are red, tender bumps that typically develop fully within three days and cause pain, redness, and tenderness of the lid margins. Regular cleaning of the eyelid margins can help minimize such conditions. Another issue is a chalazion, an inflammation of the meibomian glands inside the eyelid, which may require surgical intervention if left untreated.

Proper eyelid hygiene can minimize the severity of these problems, including rosacea and seborrhea. Cleaning the eyelashes, eyelids, or the periocular area, and can serve to reduce the presence of harmful bacteria that can cause infections or inflammation in patients.

Typical skin cleanser compositions employ strong detergents and have a pH far outside a comfortable range. Existing skin ointments are also often unsuitable for managing eyelid health due to their thick and heavy texture. They can be challenging to use around the eyes without getting into the eyes and may not permit flexible use during the day. These factors can lead to a lack of compliance with the recommended application frequency, hindering the effectiveness of the treatment.

Demodex mite overgrowth creates bacterial overgrowth, due to mite-associated microbiota, and chronic inflammatory skin conditions. Demodex folliculorum and Demodex brevis mites are implicated in chronic blepharitis and meibomian gland dysfunction. No single agent addresses all of these conditions simultaneously.

Therefore, there is a need for a suitable topical formulation that can be used to treat Demodex mite overgrowth, ocular rosacea and inflammatory skin conditions.

### SUMMARY

The present invention is a topical formulation having an ointment base and a mixture of active pharmaceutical ingredients (APIs) contained in the ointment base. API mixture consists of metronidazole, ivermectin, and Melaleuca alternifolia (tea tree) oil. The APIs are about 0.12%-6% w/w and the ointment base is about 94% to 99% w/w of the topical formulation. The formulation can further include enhancers for the APIs, such as, moisturizers and anti-oxidants. The formulation is configured as an anti-parasitic, anti-inflammatory and anti-microbial formulation.

Metronidazole is a broad-spectrum antimicrobial and anti-inflammatory agent. Ivermectin is an anti-parasitic and potent acaricidal effective against Demodex species with anti-inflammatory properties. Tea Tree Oil (Melaleuca alternifolia) is a natural essential oil with acaricidal, antibacterial, and anti-inflammatory properties. The ointment is semi-occlusive and adheres to the eyelid margin and lash follicles where Demodex mites reside to improve retention and bioavailability without dripping or migrating into the eye. The combination of the three APIs, in the ointment base, has an expected synergistic effect in the treatment of Demodex mites residing in eyelids and lashes. The semi-occlusive properties of the formulation can also be utilized in other topical areas, such as, the surface of the skin. Thus, the formulation is optimally configured for both ophthalmological and dermatological use.

### DETAILED DESCRIPTION

The term and phrases “invention,” “present invention,” “instant invention,” and similar terms and phrases as used herein are non-limiting and are not intended to limit the present subject matter to any single embodiment, but rather encompass all possible embodiments as described.

The topical formulation and method for use can “comprise,” “consist essentially of,” or “consist of” any of the ingredients or steps disclosed throughout the specification and claims. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and can include the ingredients of the present invention and do not exclude other ingredients or elements described herein. The use of the word “a” or “an” when used in conjunction with the term “comprising” in the claims and/or the specification may mean “one,” but it is also consistent with the meaning of “one or more,” “at least one,” and “one or more than one.” As used herein, “consisting essentially of” means that the invention may include ingredients in addition to those recited in the claim, but only if the additional ingredients do not materially alter the basic and novel characteristics of the claimed invention. Generally, such additives may not be present at all or only in trace amounts. However, it may be possible to include up to about 10% by weight of materials that could materially alter the basic and novel characteristics of the invention as long as the utility of the composition (as opposed to the degree of utility) is maintained.

As used herein, all weight percentages (wt. %) are based on the total wt. % of the topical formulation, unless otherwise specified. Additionally, all composition percentages are based on totals equal to 100 wt. %, unless otherwise specified. Unless otherwise indicated, percentages or concentrations refer to percentages or concentrations by weight (ie, % (w/w)). Unless otherwise specified, all ranges are inclusive, for example, “0.5 to 5” includes the 0.5 and 5 endpoints.

In an exemplary embodiment, a topical formulation (or “formulation”) includes a mixture of metronidazole, ivermectin and Melaleuca alternifolia (tea tree) oil that is incorporated into an ointment base for treatment of Demodex mites and ocular rosacea. In one exemplary embodiment, the formulation includes a mixture of about 0.01%-2% w/w metronidazole, about 0.1%-2% w/w ivermectin and about 0.01%-2% w/w Melaleuca alternifolia (tea tree) oil in about 1 g-10 g of an ointment base. In a specific embodiment, the formulation includes a mixture of 0.5% w/w metronidazole, about 1% w/w ivermectin and 0.3% tea tree oil in about 5 g of an ointment base. However, it is understood that these amounts will vary with different percentages of the APIs. In an exemplary embodiment, the APIs are about 0.12%-6% w/w of the topical formulation.

Metronidazole is an antibiotic used to treat skin infections and ocular rosacea while ivermectin is an anti-parasitic used in treating Demodex mites. Metronidazole inhibits organism protein synthesis by interacting with DNA causing organism cell death while ivermectin enhances the permeability of parasites causing paralysis and death by binding to glutamate-gated chloride channels. Tea tree oil independently exhibits potent acaricidal and anti-inflammatory properties, as demonstrated in both in vitro studies and clinical use, making it a well-established treatment option for Demodex blepharitis.

The formulation is mixed with about 94% to 99% of a suitable ointment base. In one embodiment, the ointment base is a plasticized anhydrous ointment base containing a mixture of mineral oil and polyethylene glycol to facilitate improved adhesion for external, topical use. In one or more embodiments, the ointment base is petrolatum for areas requiring a substantially higher occlusive barrier.

This novel mixture of the three active pharmaceutical ingredients-metronidazole, ivermectin and tea tree oil-in an ointment base, is expected to exert a substantially synergistic effect by combining their complementary mechanisms of action in the treatment of mite infestations on the skin or ocular surface. This rationale is supported by a comprehensive understanding of Demodex blepharitis pathophysiology and current therapeutic gaps as detailed in the inventor's comprehensive review of the disease and emerging therapies. Furthermore, the low concentration of the APIs is beneficial in reducing the likelihood of microbial resistance and irritant contact dermatitis from higher doses of each individual agent.

This formulation can be of use in the treatment of topical skin conditions, particularly rosacea, perioral dermatitis, demodicosis, and acneiform eruptions associated with Demodex mite overpopulation and bacterial pathogens.

In an embodiment, the method of preparing the formulation involves mixing metronidazole, ivermectin and tea tree oil and then incorporating the mixture into a suitable ointment base. The pH of the resulting mixture is adjusted using hydrochloric acid or sodium hydroxide solutions to the pH range of 6 to 7.5.

In another embodiment of this invention, the formulation comprises one or more moisturizers and anti-oxidants to enhance the effect of the API mixture, enhance tolerability for long-term use, and improve the outcomes.

Moisturizers are chemicals that prevent the loss of water through the skin. These moisturizers work by creating a protective film over the skin, preventing water from evaporating, or by attracting moisture from the air into the skin through hydroscopic molecules. Suitable moisturizers include, but are not limited to, ceramides, methyl gluceth-20, sorbitol, glycerine, propylene glycol, carboxylates, amino acids, glucoside derivatives, urea, lactates, and pantothenic acid derivatives such as panthenol, D-panthenol, and D, L-panthenol. Moisturizers in the formulation are each in the range of 0.05 to 20% w/w.

The anti-oxidants can be selected from the group consisting of Vitamin E and its derivatives, green tea extract, Vitamin C and its derivatives. In one specific embodiment, formulation can include Vitamin E.

The formulation is optimally configured in an ointment form. However, it can also be configured as a liquid, cream, lotion, paste, spray, mist, gel, or a foam.

An embodiment of the invention involves impregnating a therapeutically effective amount of the formulation on a substrate, such as, a wipe, a towelette, a fabric pad, or any other absorbent material. The term “pad” refers to a thick fabric that can retain the composition, and the fabric can be made of materials like rayon or cotton. Optionally, the fabric pad may have a textured surface. The impregnated substrate can be used to wipe the surface of the skin/eye, thereby treating this mite-infected surface.

The one or more embodiments of the formulation disclosed herein provide a non-invasive, easy-to-use therapeutic option for managing bacterial and inflammatory eyelid conditions, reducing the need for systemic antibiotics or steroid-based treatments. Patients who use the formulation can experience improved symptom relief, including decreased crusting, redness, swelling, and discomfort, with regular use. The topical application ensures localized efficacy while minimizing systemic absorption, reducing potential side effects associated with oral antibiotics and NSAIDs.

Thus, the formulation disclosed herein offers a significant advancement in the treatment of Demodex mites, providing healthcare providers with an effective, multi-functional solution for managing bacterial infections of the skin and inflammatory skin and ocular conditions. Beneficially, the formulation deter bacterial growth, inhibit biofilm formation, and alleviate swelling and discomfort. The unique formulation allows for both cleansing and treatment in a single step, enhancing patient compliance and therapeutic outcomes making it ideal for the targeted treatment of chronic blepharitis, ocular rosacea, and allergic irritation as part of a daily maintenance routine to prevent recurrent infection and inflammation.

Ávila et al. evaluated the efficacy of a combinatorial therapy using ivermectin and metronidazole for treating Demodex-related blepharitis (Ávila et al., Cont. Lens Anterior Eye, 2021). The study enrolled sixty patients, administering topical ivermectin (0.1%) and metronidazole (1%) to the treatment group at three time points over thirty days. Compared to the control group, which received only the topical vehicle, the treatment led to a significant elimination of Demodex mites (>96%). Additionally, the authors observed a notable reduction in inflammation and related symptoms in the treatment group, with no significant adverse effects directly attributed to the therapy. However, they acknowledged that the study lacked a comparison to single-agent treatments and an objective assessment of patient-reported symptoms.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only.

1. A topical formulation, comprising:
a. an ointment base; and
b. a mixture of active pharmaceutical ingredients (APIs) contained in the ointment base, the mixture of APIs consisting of:
i. Metronidazole;
ii. Ivermectin; and
iii. Melaleuca alternifolia (tea tree) oil,
wherein the APIs are about 0.12%-6% w/w and the ointment base is about 94% to 99% w/w of the topical formulation.

2. The topical formulation according to claim 1, comprising about 0.01%-2% w/w metronidazole.

3. The topical formulation according to claim 1, comprising about 0.1%-2% w/w ivermectin.

4. The topical formulation according to claim 1, comprising about 0.01%-2% w/w Melaleuca alternifolia (tea tree) oil.

5. The topical formulation according to claim 1, further comprising about 1 g-10 g of the ointment base.

6. The topical formulation according to claim 1, wherein the ointment base comprises a petrolatum ointment base.

7. The topical formulation according to claim 1, wherein the ointment base is a plasticized anhydrous ointment base.

8. The topical formulation according to claim 7, wherein the plasticized anhydrous ointment base contains a mixture of mineral oil and polyethylene glycol.

9. The topical formulation according to claim 1, further comprising one or more moisturizers.

10. The topical formulation according to claim 1, wherein the one or more moisturizers is selected from the group consisting of ceramides, methyl gluceth-20, sorbitol, glycerine, propylene glycol, carboxylates, amino acids, glucoside, urea, lactates, pantothenic acid and derivatives and mixtures thereof.

11. The topical formulation according to claim 1, further comprising one or more anti-oxidants.

12. The topical formulation according to claim 9, where at least one of the one or more anti-oxidants is selected from the group consisting of Vitamin E and its derivatives, green tea extract, Vitamin C and its derivatives.

13. The topical formulation according to claim 1, wherein the topical formulation is configured as an anti-parasitic, anti-inflammatory and anti-microbial formulation.

14. The topical formulation according to claim 1, wherein the pH of the formulation is around 6 to 7.5.

15. The topical formulation according to claim 1, wherein the formulation is configured for ophthalmological and dermatological use.
